(19) United States
(12) Patent Application Publication
(10) Pub. No.: US 2009/0246888 A1
(43) Pub. Date: Oct. 1, 2009

(54) NANOASSAYS

(75) Inventors: Larry J. Kricka, Devon, PA (US); Jason Y. Park, Philadelphia, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: The Trustees of the University of Pennsylvania, Philadelphia, PA (US)

(21) Appl. No.: 11/912,583

(22) PCT Filed: Apr. 26, 2006
(86) PCT No.: PCT/US06/16250

§ 371 Date: May 14, 2008

Related U.S. Application Data
(60) Provisional application No. 60/675,213, filed on Apr. 27, 2005.
(60) Provisional application No. 60/736,985, filed on Nov. 15, 2005.

(51) Int. Cl. G01N 33/543; G01N 21/64; C12M 1/00; B05D 3/00

(52) U.S. Cl. 436/518; 422/82.08; 435/287.2; 427/2.13

(57) ABSTRACT

The present invention provides assays of nanometer-level dimension.

### CROSS-REFERENCE TO RELATED APPLICATIONS

This application claims priority to U.S. Provisional Application Ser. No. 60/736,985 filed Nov. 15, 2005, and U.S. Provisional Application Ser. No. 60/675,213 filed Apr. 27, 2005, each of which is incorporated herein by reference in its entirety.

### FIELD

The present invention provides assays of nanometer-level dimension.

### BACKGROUND

Dip-pen nanolithography (DPN) is a scanning probe high-resolution nanopatterning technique in which an atomic force microscopy (AFM) tip coated with molecules is used to deliver molecules via capillary transport from the tip to a surface via a solvent meniscus. DPN provides a resolution of about 10 nm. DPN has been used to construct arrays of protein with 100 to 350 nanometer features. DPN provides higher resolution than some of the other conventional patterning techniques that have been used to construct protein arrays, such as photolithography, micro-contact printing, and spot arraying. Although nanopatterning techniques such as DPN have been used to create binding assays, the full potential of these techniques have not been realized. A need exists for improving the efficiency of binding assays by creating patterning techniques. The present invention is directed to this, as well as other, important ends.

### BRIEF DESCRIPTION OF THE DRAWINGS

FIG. 1 is a nanoarray of anti-PSA antibodies showing binding to PSA and to a PSA-alpha1-antichymotrypsin complex and expected heights as measured by AFM.

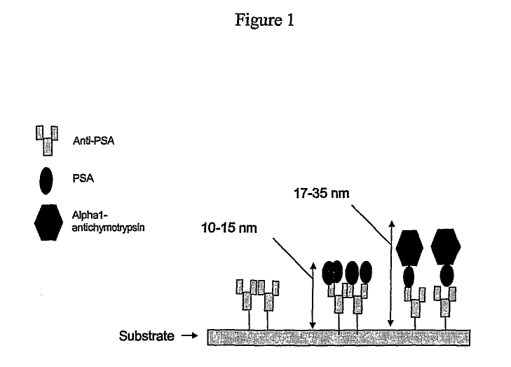

FIG. 2 is a nanoarray of enhancing polymer and anti-PSA antibodies. Polymers create a nanoenviroment that enhances the light signal from the alkaline phosphatase catalyzed decomposition of a dioxetane substrate.

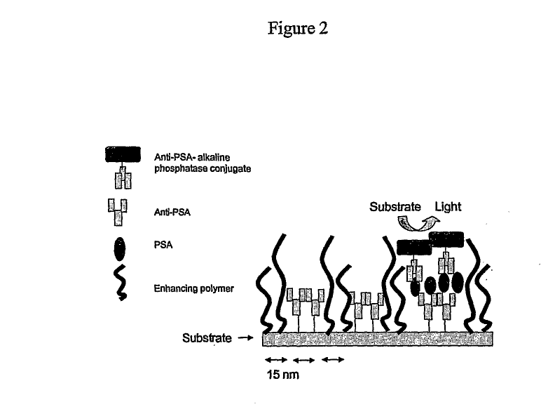

FIG. 3 is a nanoassay device based on an array of dyes and a member of a ligand:binder pair attached to a substrate using DPN. The energy transfer principle of this assay is based on steric disruption of the energy transfer fluorescence due to specific binding between the ligand and binder on the substrate surface.

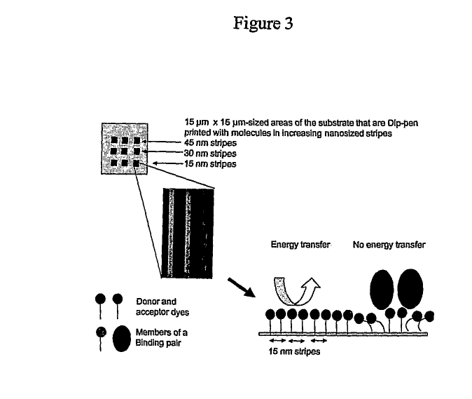

FIG. 4 is an nanoassay device based on an array of dyes and a member of a ligand:binder pair attached to a substrate using DPN. The energy transfer principle of this assay is based on steric disruption of the energy transfer fluorescence due to specific binding between the ligand and binder on the substrate surface. The energy transfer distance is increased by silver island film.

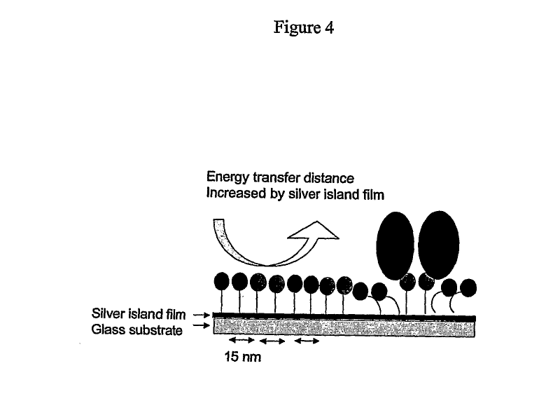

### SUMMARY

The present invention provides, inter alia, assays of nanometer-level dimension, including immunoassays, nucleic acid assays, and receptor assays. In particular, the present invention provides for the patterning of at least two patterns of different molecules in predetermined and preselected patterns on a solid surface so as to place the molecules in conformed physical relationship to each other. The solid surface can be porous or non-porous.

In one aspect of the present invention, a solid surface having at least two patterns of different molecules on the surface is provided. At least one of the patterns has a dimension between about 1 and about 1000 nanometers, preferably between about 1 and about 500 nanometers or between about 10 and about 500 nanometers.

Methods of patterning molecules on solid surfaces are known in the art. It has not been known heretofore to pattern at least two different molecules, molecules that otherwise would be free in solution, in at least two different patterns on a solid surface. By immobilizing the different molecules in at least two different patterns on a solid surface, a modified nanoenviroment is created that can enhance reaction sensitivity.

In one embodiment of the present invention, interaction between molecules of the different patterns on the solid substrate is modulated in the presence of an analyte. For example, in one aspect, the two different molecules immobilized separately, but patternwise, on the solid surface, are members of an energy transfer pair. Presence of an analyte, e.g., in a solution in contact with the surface, gives rise to a change in energy transfer between the patterned molecules.

Energy transfer can occur, for example, through fluorescence resonance energy transfer or bioluminescence energy transfer. Fluorescence resonance energy transfer occurs when part of the energy of an excited donor is transferred to an acceptor fluorophore which re-emits light at another wavelength or, alternatively, to a quencher group that typically emits the energy as heat.

In a fluorescence energy transfer pair, it is generally preferred that an absorbance band of the acceptor substantially overlap a fluorescence emission band of the donor. When the donor (e.g., fluorophore) is a component of a probe that utilizes fluorescence resonance energy transfer, the donor moiety and the quencher (acceptor) are preferably selected so that the donor and acceptor moieties exhibit fluorescence resonance energy transfer when the donor moiety is excited. One factor to be considered in choosing the fluorophore-quencher pair is the efficiency of fluorescence resonance energy transfer between them. Preferably, the efficiency of energy transfer between the donor and acceptor moieties is at least 10%, more preferably at least 50% and even more preferably at least 80%.

There is a great deal of practical guidance available in the literature for selecting appropriate donor-acceptor pairs for particular probes, as exemplified by the following references: Pesce et al., Eds., Fluorescence Spectroscopy (Marcel Dekker, New York, 1971); White et al., Fluorescence Analysis: A Practical Approach (Marcel Dekker, New York, 1970); and the like. The literature also includes references providing exhaustive lists of fluorescent and chromogenic molecules and their relevant optical properties, for choosing reporter-quencher pairs (see, for example, Berlman, Handbook of Fluorescence Spectra of Aromatic Molecules, 2nd Edition (Academic Press, New York, 1971); Griffiths, Colour and Constitution of Organic Molecules (Academic Press, New York, 1976); Bishop, Ed., Indicators (Pergamon Press, Oxford, 1972); Haugland, Handbook of Fluorescent Probes and Research Chemicals (Molecular Probes, Eugene, 1992) Pringsheim, Fluorescence and Phosphorescence (Interscience Publishers, New York, 1949); Wu, P. and Brand, L. (1994) Anal Biochem. “Resonance energy transfer: methods and applications.” 218, 1-13 and the like. Further, there is extensive guidance in the literature for derivatizing acceptor and quencher molecules for covalent attachment via readily available reactive groups that can be added to a molecule.

Fluorophore donor and acceptor combinations are well known in the art and include, for example, the Alexa Fluor dye combinations from Molecular Probes, Invitrogen Detection Technologies; BODIPY dye combinations from Molecular Probes (Eugene, Oreg.); donor fluorescein and acceptors 6-carboxy-X-rhodamine (Applied Biosystems, Foster City, Calif.), N,N,N′,N′-tetramethyl-6-carboxy-rhodamine (Applied Biosystems, Foster City, Calif.), rhodamine, or Texas Red; and donor cascade blue with acceptor fluorescein.

Many binding assays, especially immunoassays for clinically important analytes, have been developed based on modulation of molecular interactions in solution or at a solid-liquid interface. An important type exploits the modulation of energy transfer between two fluorescent dyes, a fluorescence energy donor and acceptor. The assay design is as shown below for an antigen (Ag) and an antibody (Ab) format:

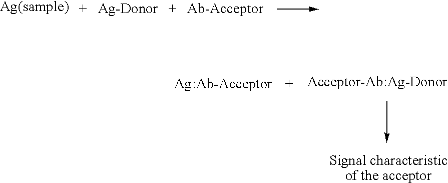

In this assay a sample is incubated with an antigen labeled with a donor (e.g., fluorescein) and an antibody specific for the antigen labeled with an acceptor (e.g., tetramethylrhodamine). After incubation the reaction mixture is exposed to excitation light specific for the donor fluorophore. If a complex has formed (acceptor-Ab:Ag-donor) then the incident radiation excites the donor that then transfers energy to the acceptor; the acceptor is in close proximity by virtue of the formation of the complex. The acceptor then emits at the frequency characteristic of the Acceptor. Measurement of this light emission provides an indication of the amount of Ag in the sample. Low Ag concentration will lead to a large signal from the acceptor dye whereas high Ag concentration leads to a low signal because the complex is formed in very low concentration or not at all.

Energy transfer is proportional to the sixth power of the distance between the donor and acceptor dyes. Energy transfer is characterized by the distance at which energy transfer is 50% efficient (the Forster distance). For an ideal donor acceptor pair, this distance is 8.4 nm (84 Angstroms). In solution, this distance is difficult to control due to molecular motion of the molecules to which the dyes are attached but nevertheless effective assays are possible based on this principle. Many types of assay that rely on molecular interaction could be rendered more efficient if the molecules were specifically patterned on a surface using nanolithographic printing.

For example, in the case of an exemplary energy transfer assay of the present invention, the substrate or solid surface is patterned with donor and acceptor dyes in separate precisely controlled patterns that maximize energy transfer. Also patterned on to the solid surface is a binding agent adjacent to the donor and acceptor dyes. Binding of a specific analyte to the binding agent disrupts the energy transfer and signals the presence of the analyte in a sample. This can form the basis of a simple dip-stick type of assay for an analyte that involves exposing a substrate patterned with donor dyes, acceptor dyes and a binding agent in separate precisely controlled patterns, and then assessing the color of the fluorescence from the patterned substrate when irradiated with excitation light for the donor. For a sample containing low concentrations of the analyte, the observed fluorescence would be characteristic of the acceptor due to effective energy transfer. A sample with a high concentration of analyte would disrupt the energy transfer and the observed fluorescence would be characteristic of the donor. For use herein, the term adjacent means close in proximity or nearby.

In some embodiments of the present invention, silver island films can be used to improve the energy transfer between the donor and acceptor dyes. Silver island films are thin films of silver that have islands of silver particles distributed across the film. The films can be prepared by reducing silver ions by glucose on clean quartz slides (Lakowicz et al., 2001, 2002) or by evaporation in a vacuum chamber at a pressure below 10⁻³ Pa.

It has been shown that resonance energy transfer distances can be increasing by placing donor- and acceptor-labeled DNA oligomers between two slides coated with metallic silver particles. The Forster distance increased from 58 to 77 Å. Other studies have shown that proximity of fluorophores to silver islands results in increased fluorescence intensity, with the largest enhancement for the lowest-quantum-yield fluorophores (Lakowicz et al., 2003; Malicka et al., 2003).

Accordingly, in one aspect of the present invention, a donor fluorophore and an acceptor fluorophore are separately patterned on a solid surface. The geometry and identity of the fluorophores is such that they interact with each other to generate a detectable signal. Interspersed between the donor and acceptor fluorophore is a binding agent. Energy transfer between the two fluorophores is disrupted when an analyte binds to the binding agent thereby modulating the interaction between the fluorophores. In some embodiments, the fluorophores are placed on slides coated with metallic silver particles., e.g., silver island films.

Any molecules that have an interaction that is modulated in the presence of an analyte can be patterned on a solid surface can be used in an assay of the present invention. Examples of assays that rely on molecular interactions that can be adapted for use in the present invention can be found in Price C. P. and Newman D. J. (eds) Principles and Practice of Immunoassay (Stockton Press, New York, 1997) and Wild D. (ed) The Immunoassay Handbook (Elsevier, San Diego, 2005), each of which is hereby incorporated by reference in its entirety and for all purposes. Examples include drug-enzyme conjugates and drug-enzyme donor conjugates such as those used in enzyme multiplied immunoassays (EMIT) and CEDIA assays

Emit is a homogeneous enzyme immunoassay that is also very widely used in clinical analyses. Because EMIT does not require a separation step, it is simple to perform and has been used to develop a wide variety of drug, hormone, and metabolite assays (Rubenstein K E, Schneider R S, Ullman E F. “Homogeneous” enzyme immunoassay: New immunochemical technique. Biochem Biophys Res Commun 1972; 47:846-851.) Due to their operational simplicity, EMIT-type assays are easily automated and are included in the repertoire of many automated clinical and immunoassay analyzers. In this technique, antibody against the analyte drug, hormone, or metabolite is added together with substrate to the patient's sample. Binding of the antibody and analyte occurs. An aliquot of the enzyme conjugate of the analyte drug, hormone, or metabolite is then added as a second reagent; the enzyme-analyte conjugate then binds with the excess analyte antibody, forming an antigen-antibody complex. This binding of the analyte antibody with the enzyme-analyte conjugate affects enzyme activity by physically blocking access of the substrate to the active site of the enzyme or by changing the conformation of the enzyme molecule and thus altering its activity. To complete the assay, the resultant enzyme activity is measured. The relative change in enzyme activity resulting from the formation of the antigen-antibody complex is proportional to the drug, hormone, or metabolite concentration in the patient's sample. Concentration of the analyte is calculated from a calibration curve prepared by analyzing calibrators that contain known quantities of the analyte in question.

Enzyme multiplied immunoassays can be adapted for use in the methods and assays of the present invention. For example, for use in the methods and assays of the present invention, the drug enzyme conjugate and the antibody can be separately patterned on a solid surface. The drug enzyme conjugate and antibody will be printed on the solid surface in such a way that they interact with each other. This interaction affects enzyme activity by physically blocking access of the substrate to the active site of the enzyme or by changing the conformation of the enzyme molecule thus altering its activity. The solid surface is contacted with a sample. If antigen is present in the sample, the antigen will react with the antibody thereby freeing the drug enzyme conjugate. The relative change in enzyme activity resulting from the formation of the antigen-antibody complex is proportional to the analyte concentration in the patient's sample.

CEDIA is a second type of homogeneous enzyme immunoassay. Inactive fragments (the enzyme donor and acceptor) of β-galactosidase are prepared by manipulation of the Z gene of the lac operon of E. coli. These two fragments spontaneously reassemble to form active enzyme even if the enzyme donor is attached to an antigen. However, binding of antibody to the enzyme donor-antigen conjugate inhibits reassembly, and no active enzyme is formed. Thus, competition between antigen and the enzyme donor-antigen conjugate for a fixed amount of antibody in the presence of the enzyme acceptor modulates the measured enzyme activity (high concentrations of antigen produce the least inhibition of enzyme activity; low concentrations produce the greatest inhibition).

CEDIA can also be adapted for use in the methods and assays of the present invention. For example, for use in the methods and assays of the present invention, the antibody and antigen linked to an enzyme donor can be separately patterned on a solid surface. The antibody and antigen linked to the enzyme donor will be printed on the solid surface in such a way that they interact with each other. Binding of the antibody to the enzyme donor-antigen conjugate inhibits reassembly of the enzyme donor into an active enzyme. The solid surface is contacted with a sample containing enzyme acceptor. If the antigen is present in the sample, it will bind to the antibody patterned on the surface freeing up the enzyme donor to interaction with the enzyme acceptor. Competition between antigen and the enzyme donor-antigen conjugate for a fixed amount of antibody in the presence of the enzyme acceptor modulates the measured enzyme activity.

The term “analyte” as used herein refers to the substance to be detected that may be present in the sample. The analyte can be any substance for which there exists a naturally occurring specific binding member (such as an antibody or antigen), or for which a specific binding member can be prepared. The analyte, or portion thereof, can be antigenic or haptenic having at least one determinant site, or can be a member of a naturally-occurring binding pair, e.g., carbohydrate and lectin, hormone and receptor, complementary nucleic acids (e.g., DNA, RNA, mRNA) and the like. Analytes of particular interest include DNA, RNA, antigens, antibodies, proteins, peptides, carbohydrates, polysaccharide, glycoprotein, haptens, drugs, hormones, hormone metabolites, macromolecules, toxins, bacteria, viruses, enzymes, tumor markers, nucleic acids, and the like, although other types of substances can also be detected.

The phrase “specifically binds to” or “having binding specificity” when referring to a binding reaction, refers to a binding reaction which is determinative of the presence of a target analyte in the presence of a heterogeneous population of proteins and other biologics. A binding agent having binding specificity for an analyte of interest is capable of binding directly or indirectly to the analyte with a high affinity. In an exemplary embodiment, binding can be by covalent bonding, ionic bonding, ion pairing, electrostatic interaction, van der Waals association and the like. In embodiments, wherein a binding agent imparts the binding specificity, the binding moiety can be a specific binding substance capable of binding directly or indirectly to the analyte with a high affinity. The binding agent is preferably substantially free from cross-reactivity with other substances that may be present in the sample or the assay reagents. The binding agent can be a detector probe such as an oligonucleotide, peptide, ligand, antibody, antigen, or other small molecule that directly binds the analyte of interest.

While the binding agent in some embodiments will bind directly to the analyte, the present invention contemplates indirect binding of the binding moiety to the analyte, i.e., the use of one or more intermediate binding substances to sequester or effect a linkage to the analyte. For example, in some embodiments, the binding agent will bind to its binding partner which is itself bound to a detector probe bound to the analyte of interest. For example, in embodiments wherein the analyte is RNA or DNA, the binding agent can be a binding agent such as streptavidin bound to a biotinylated oligonucleotide complementary to the RNA or DNA sequence of interest. A wide variety of indirect binding protocols are available and well described in the scientific and patent literature. The term “binding agent” as used in the specification and claims are thus intended to include all substances which are able to bind the analyte, either directly (i.e., without an intermediate binding substance) or indirectly (i.e., with one or more intermediate binding substances forming a linkage).

In another aspect of the present invention, one of the molecules patterned on the solid surface modulates a signal generated by binding of an analyte to a second molecule separately patterned on the solid substrate. For example, in one aspect, the modulator molecule is a polymer that enhances a detectable signal generated in a bioluminescent or chemiluminescent assay for detecting binding of an analyte to a binding agent. The binding agent is the second molecule patterned on the solid substrate and is patterned on the surface so that molecules of polymer are adjacent to molecules of binding agent.

Chemiluminescent and bioluminescent reactions are known in the art. For example, chemiluminescent assays based on adamantyl 1,2-dioxetane aryl phosphates are one of the principal types of assays for detecting and measuring the activity of alkaline phosphatase. They are widely used in high throughput clinical immunoassay analyzers and commercial Western blotting kits for proteins and Southern and Northern blotting kits for nucleic acids. Alkaline phosphatase cleaves the phosphate group to produce an unstable phenoxide. This decomposes via scission of the 1,2-dioxetane ring to produce adamantanone and a fluorescent phenoxy ester in an electronically excited state. The latter intermediate decays to the electronic ground state and the excess energy is emitted as a glow of light (Bronstein et al. J Biolumin Chemilumin 1989; 4:99-111).

Chemilummescent assays based on the luminol reaction enhanced by phenols are one of the principal types of assays for detecting and measuring the activity of horseradish peroxidase and are widely used in high throughput routine clinical immunoassay analyzers and commercial Western blotting kits for proteins and Southern and Northern blotting kits for nucleic acids.

The gene for firefly luciferase has become important as a reporter gene (Bronstein et al., Biotechniques 1994; 17:172-4, 176-7), and gene expression is assessed by measuring the activity of the expressed firefly luciferase using a mixture of Mg-ATP and firefly luciferin.

A wide variety of substrates (e.g., luminescent compounds) have been identified in the art for use with luminescent assays. These include, but are not limited to, 1,2-dioxetanes, cyclic diacylhydrazide compounds, and luciferin for use with enzymes such as phosphatases (e.g., alkaline phosphatase), peroxidases (e.g., horseradish peroxidase) and luciferases (e.g., firefly luciferase).

Dioxetanes are compounds having a 4-membered ring in which 2 of the members are oxygen atoms bonded to each other. Dioxetanes can be thermally or photochemically decomposed to form carbonyl products, e.g., ketones or aldehydes. Release of energy in the form of light (i.e. luminescence) accompanies the decompositions. The dioxetanes can be used in an assay method in which a member of a specific binding pair (i.e. two substance that bind specifically to each other) is detected by means of an optically detectable reaction. According to this method, the dioxetane is contacted with an enzyme that causes the dioxetane to decompose to form a luminescent substance (i.e. a substance that emits energy in the form of light). The luminescent substance is detected as an indication of the presence of the first substance. By measuring, for example, the intensity of luminescence or the total amount of luminescence, the concentration of the first substance can be determined. Where the enzyme is an oxido-reductase (preferably a peroxidase, e.g., horseradish peroxidase or microperoxidase), it causes the dioxetane to decompose by cleaving the 0-0 bond of the 4-membered ring portion of the dioxetane. The enzyme can act directly on the dioxetane substrate or can be mediated through the addition of peroxide. Where the dioxetane includes an enzyme cleavable group (e.g., phosphate), the enzyme (e.g., phosphatase) causes the dioxetane to decompose by cleaving the enzyme cleavable group from the dioxetane. Cleavage yields a negatively charged atom (e.g., an oxygen atom) bonded to the dioxetane, which in turn destabilizes the dioxetane, causing it to decompose and emit radiation, which in turn is absorbed by the portion of the molecule containing the fluorescent chromophore, which consequently luminescence.

1,2-dioxetanes are well established in the art. Suitable dioxetanes are for example those disclosed in U.S. Pat. Nos. 4,978,614; 4,952,707; 5,089,630; 5,112,960; 5,538,847; 4,857,652; 5,849,495; 5,547,836; 5,145,772; 6,287,767; 6,132,956; 6,410,751; 6,353,129; 6,284,899; 6,245,928; 6,180,833; 5,892,064; 5,886,238; 5,866,045; 5,578,523; each of which is incorporated by reference herein in its entirety and for all purposes. In some embodiments, a hydrophobic fluorometric substrate is used in conjunction with the 1,2-dioxetane. A hydrophobic fluorometric substrate is a compound which upon activation by an enzyme can be induced to emit in response to energy transfer from an excited state dioxetane decomposition product donor. As the donor is hydrophobic, the substrate, when activated, must be sufficiently hydrophobic as to be sequestered in the same hydrophobic regions to which the donor migrates, for energy and transfer to occur. Exemplary fluorometric substrates are AttoPhos™ and AttoPhos Plus™ invented by JBL Scientific Inc. and distributed by Promega.

In general, any chemiluminescent dioxetane which can be caused to decompose and chemiluminesce by interaction with an enzyme can be used in connection with this invention. Suitable dioxetanes are available from commercial sources such as the AMPPD™, CSPD™, CDP™, and CDP™-Star substrates marketed by Tropix (Bedford, Mass.) and Lumigen PPD™, Lumi-Phos™, Lumi-Phos 530 ™, and Lumi-Phos Plus™, available from Lumigen Inc. (Southfield, Mich.).

Typically, the 1,2-dioxetanes useful in this invention will have the general formula:

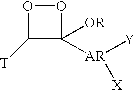

In these 1,2-dioxetanes, T is a stabilizing group. Because the dioxetane molecule, without the stabilizing group, may spontaneously decompose, a group, typically a polycycloalkyl group is bound to the dioxetane to stabilize it against spontaneous decomposition. This need for stabilization has resulted in commercially developed 1,2-dioxetanes being generally spiroadamantyl. The adamantyl group, spiro-bound, can be optionally substituted at any bridge head carbon, to affect chemiluminescent properties. As indicated, the remaining carbon of the dioxetane ring bears a OR substituent, wherein R is generally an alkyl or cycloalkyl, although it may be a further aryl group. The alkyl can be optionally substituted, with the substituent including halogenated groups, such as polyhaloalkyl substituents. The remaining valence is occupied by an aryl moiety, preferably phenyl or naphthyl. If naphthyl, particular substitution profiles on the naphthyl ring are preferred. The aryl ring bears at least one substituent, X. In commercially developed dioxetanes, this is typically an enzyme-cleavable group. Where the associated enzyme is alkaline phosphatase, for example, the enzyme-cleavable group X will be a phosphate. The aryl ring may also bear a substituent Y, which is selected to be either electron donating, or electron withdrawing. Preferred groups include chlorine, alkoxy and heteroaryl, although other groups may be employed. These substitutions can further effect chemiluminescent properties, and reaction kinetics. A wide variety of other substituents are disclosed in the referenced patents.

A class of compounds receiving particular attention with respect to luminescent reactions utilizing a peroxidase enzyme, e.g., horseradish peroxidase, are dihydrophthalazinedione compounds that are used in combination with an oxidant, preferably a peroxide compound such as hydrogen peroxide. Any chemiluminescent dihydrophthalazinedione can be used as substrate in the present invention, that is to say any dihydrophthalazinedione which is oxidisable in the presence of a peroxidase catalyst by an addition of an oxidant to give chemiluminescence. Dihydrophthalazinediones are well established in the art. Suitable dihydrophthalazinediones as well as other compounds for use with peroxidases, (e.g., acridinium compounds, such as acridinium esters and benzacridinium, and alkenes) are, for example, those disclosed in U.S. Pat. Nos. 5,552,298; 6,696,569; 6,410,732; 5,922,558; 5,750,698; 5,723,295; 5,670,644; 5,601,977; 5,552,298; 5,523,212; 5,879,894; 6,635,437; 6,296,787; 6,270,695; 6,218,137; 6,139,782; 6,126,870; 6,045,991; 5,965,736; 5,840,963; 5,772,926; and 5,686,258; each of which is incorporated herein by reference in its entirety. Preferred dihydrophthalazinediones include substituted aryl cyclic diacylhydrazide including aminoaryl cyclic diacylhydrazides such as luminol, isoluminol, aminobutylethylisoluminol, aminoethyl-ethylisoluminol and 7-dimethylaminonaphthalene-1,2-dicarboxylic acid hydrazide and hydroxyaryl cyclic diacylhydrazides, for example, 5-hydroxy-2,3-dihydro-phthalazine-1,4-dione; 6-hydroxy-2,3-dihydro-phthalazine-1,4-dione; 5-hydroxy-2,3-dihydro-benzo[g]phthalazine-1,4-dione; and 9-hydroxy-2,3-dihydro-benzo[f]phthalazine-1,4-dione. Peroxide compounds include hydrogen peroxide, sodium perborate, urea peroxide, and the like.

The sensitivity of the peroxidase-catalyzed chemiluminescent oxidation of dihydrophthalazinediones can be enhanced by including an enhancer in the reaction. The enhancer will be present in an amount which enhances light production from the diacylhydrazide in the presence of the peroxidase and/or decreases background chemiluminescence. Enhancers are known in the art and include, phenolic compounds such as those disclosed in U.S. Pat. No. 5,306,621, incorporated herein by reference in its entirety, including p-phenylphenol, p-iodophenol, p-bromophenol, p-hydroxycinnamic acid 6-bromo-2-naphthol, D-luciferin, and 2-cyano-6-hydroxybenzothiazole as well as boronic compounds, such as those disclosed in U.S. Pat. No. 5,629,168, incorporated herein by reference in its entirety, including, 4-iodophenylboronic acid (PIBA), 4-bromophenylboronic acid (PBBA), 4-chlorophenylboronic acid, 3-chlorophenylboronic acid, 3,4-dichlorophenylboronic acid, 2,3-dichlorophenylboronic acid, 5-bromo-2-methoxybenzeneboronic acid, 3-nitrophenylboronic acid, 4-chloro-3-nitrophenylboronic acid, 3-aminophenylboronic acid, 3-amino-2,4,6-trichlorophenylboronic acid, 4-(2′-carboxyethenyl)phenylboronic acid, 1-naphthaleneboronic acid, 6-hydroxy-2-naphthaleneboronic acid, phenylboronic acid, 2-methylphenylboronic acid, 4-methylphenylboronic acid, dimethyl-phenylboronic acid, 4-bromophenyl-di-n-butoxyborane, 4-carboxy-3-nitrophenylboronic acid, 4-(trimethylsilyl)benzeneboronic acid, 4-biphenylboronic acid, 4-(phenoxy)benzeneboronic acid, 4-(3′-borono-4′-hydroxyphenylazo)benzoic acid, diphenylisobutoxyborane, 4-(4′-chloroanilino)phenylboronic acid, 4,4′-bis(phenylboronic acid), 4-(4′-bromophenyl)phenyl-di-n-butoxyborane, di(3′,5′-dichlorophenoxy)-3,5-dichlorophenylborane, 4-chlorophenyl-di-(4′-chlorophenoxy)borane, pentaerythritol borate, boroglycine, 2-phenyl-1,3,2-dioxaborinane, bis(catechol)borate and 2-hydroxy-5-[(3′-trifluoromethyl)phenylazo]benzeboronic acid and diphenylboronic anhydride. Other enhancers include 6-hydroxybenzothiazole, substituted phenols, such as those disclosed in U.S. Pat. No. 4,598,044, incorporated herein by reference in its entirety; aromatic amines including those disclosed in U.S. Pat. No. 4,729,950, incorporated herein by reference in its entirety; and phenols substituted in ortho and/or para positions by imidazolyl or benzimidazolyl (U.S. Pat. No. 5,043,266, incorporated herein by reference in its entirety).

In some embodiments, the bioluminescent assay will use a luciferase enzyme. Examples are luciferases isolated from a variety of luminous organisms, such as the luciferase genes of Photinus pyralis (the common firefly of North America), Pyrophorus plagiophthalamus(the Jamaican click beetle), Renilla reniformis (the sea pansy), and several bacteria (e.g., Xenorhabdus luminescens and Vibrio spp). Luciferases are enzymes found in luminous organisms which catalyze luminescence reactions. They are organized into groups based on commonalties of their luminescence reactions. All luciferases within a group are derived from related luminous organisms, and all catalyze the same chemical reaction. Examples are beetle luciferases, which all catalyze ATP-mediated oxidation of the beetle luciferin; and anthozoan luciferases which all catalyze oxidation of coelenterazine (Ward WW and Cormier MJ. Proc Natl Acad Sci 1975; 72(7):2530-4.). With the technical capabilities of molecular biology, it is possible to alter the structure of a luciferase found in nature to yield a functional equivalent thereof. A functional equivalent is an enzyme that maintains the ability to catalyze the same luminescence reaction, and thus it remains in the same group of enzymes. Luciferase as used herein is intended to include naturally occurring and non-naturally occurring luciferase enzymes.

Luciferases generate light via the oxidation of enzyme-specific substrates, called luciferins. For firefly luciferase and all other beetle luciferases, this is typically done in the presence of magnesium ions, oxygen, and ATP. For anthozoan luciferases, including Renilla luciferase, oxygen is required along with the luciferin. Additional reagents such as, for example, coenzyme A can be used to yield greater enzyme turnover and greater luminescence intensity.

It will be understood that other molecules that generate light by interaction with chemiluminescent reactants can be used in the present invention such as hemin which interacts with reactants such as luminol and peroxide and aequorin which interacts with calcium ion to generate luminescence.

The performance of luminescent reactions can be improved by use of binding assays of the present invention. Although polymer enhancement of luminescent enzyme catalyzed reactions is known, the use of polymer immobilized on a solid surface adjacent to a binding agent or two fluorophores, or other interacting molecules, immobilized on a solid surface as described herein has not been known heretofore.

The term “polymer”, as used herein, refers to molecules formed from the chemical union of two or more repeating units. Accordingly, included within the term “polymer” may be, for example, dimers, trimers and oligomers. The polymer may be synthetic, naturally-occurring or semisynthetic. Any polymer that can modulate, e.g., enhance a signal from a luminescent reaction can be used in the present invention. Preferably the polymer will provide a more hydrophobic environment. Polymers and other materials for use in the present invention can include for example, materials that can be converted into nanofibers, such as, for example, poly(lactic acid-co-glycolic acid), poly(acrylic acid)-poly(pyrene methanol), sodium citrate, polypyrrole, poly (3-methylthiophene), polyaniline, polyacrylonitrile, poly(p-phyenylene), poly(3,4-ethylenedioxythiophene), polyacrylonitrile, poly(L-lactic acid)-polycaprolactone, blends, polystyrene-block-poly(2-cinnamoylethyl methacrylate), polystyrene-block-poly(2-cinnamoylethyl methacrylate)-block-poly(tert-butyl acrylate), peptide-amphiphile, dendrimer, bolaform glucosamide; materials that can electrospun into nanofibers, such as for example, polystyrene, polycarbonate, polymethacrylate, polyvinylchloride, polyethylene terephthalate, nylon6,6, nylon4,6, polyamide, polyurethanes, polyvinyl alcohol, polylactic acid, polycaprolactone, polyethylene glycol, polylactide-co-glycolide, polyethylene-co-vinyl acetate, polyethylene co-vinyl alcohol, polyethylene oxide, collagen; amphiphilic poly(2-methyloxazoline-block-dimethylsiloxane-block-2-methyloxazoline)(PMOXA-b-PDMS-b-PMOXA) ABA triblock copolymers; poly(thiophene); polyetherketone; polyallylamine; polyethyleneimine; poly(iminohexamethylene); polytetrafluoroethylene; poly(oxy-1,4,-phenyleneoxyl-1,4-phenylenecarbonyl-1,4-phenylene); polyvinylidene fluoride; polymethyl methacrylate; polystyrene; aluminum; palladium; silicon; or blends or composites thereof.

Although any of the polymers disclosed herein can be used in connection with any of the chemi- or bio-luminescent reactions, certain polymers will be preferred in combination with certain labels. For example, for use with alkaline phosphatase catalyzed reactions, preferred polymers to be immobilized on the nanostructures include polyhydroxyacrylates, polyvinylalkylethers, polyvinyl alcohol, polyvinylalkylpyrrolidinones, BSA, nylon, and poly[vinylbenzyl(benzyldimethyl ammonium) chloride]. While not wishing to be bound by any particular theory, it is postulated that the role of the polymer is to provide a more hydrophobic environment for decomposition of the excited electronic state intermediate formed in the scission of the 1,2-dioxetane ring structure. The polymer can be optionally combined with fluorophores that then act as energy acceptors and emit light at a wavelength characteristic of the fluorophore. This effect can be seen, for example, with enzyme in solution or immobilized as part the detection conjugate in a sandwich assay. In both situations, the enzyme is a dynamic environment and interaction between the polymer and the enzyme or the products of the enzyme reaction are governed by the random motion of the polymer in solution. In an exemplary embodiment of the present invention, the polymer and binding agent immobilized on the solid surface, on a nanometer scale, or two members of an energy transfer pair immobilized on the solid surface, on a nanometer scale, will create a more ordered and/or more static nanoenvironment that will maximize energy transfer in a luminescent reaction. The enhancement effect can be improved by a tighter control of the nanoenvironment as provided by the present invention.

For use with peroxidase catalyzed reactions, preferred polymers to be immobilized on the nanostructures include hydroxypropyl methylcellulose, hydroxyethyl cellulose, and hydroxybutyl methylcellulose. Boronic or phenolic enhancers are generally used in combination with the horseradish peroxidase and its substrates. A polysorbate, such as Tween 20 can also be used to stabilize light emission from the horseradish peroxidase (HRP) catalyzed chemiluminescent oxidation of hydroxyaryl cyclic diacylhydrazides.

For use with luciferase catalyzed reactions, preferred polymers to be immobilized on the nanostructures include polyethylene glycol and polyvinylpyrrolidone. While not wishing to be bound by any particular theory, it is postulated that the polymer is acting as a reservoir for the inhibitory oxyluciferin product and the reaction and thus constantly regenerating active firefly luciferase.

Nanolithographic printing is particularly useful for the preparation of the assays of the present invention having nanoscopic features. Any type of nanolithographic printing method can be used in the present methods to pattern at least two patterns of different molecules in predetermined and preselected patterns on a solid surface. Methods include, for example, photolithography, E-beam lithography, molecular beam ionization, and the like. These methods are known in the art and are thus not described herein in detail.

In one exemplary embodiment, dip pen nanolithography is used to pattern the molecules on the solid substrate. For example, in certain embodiments, the present invention provides two-dimensional molecular patterns fabricated with nanometer control on the surface of a substrate using a molecular printing technique, such as dip-pen nanolithography. Methods for patterning molecules using dip pen nanolithography are known in the art. See for example, US Publication Numbers 20020063212 and 20030068446, incorporated herein by reference in their entirety and for all purposes.

In dip-pen nanolithography, typically, the different patterns of molecules are formed by a plurality of dots or a plurality of lines formed on the solid surface. The plurality of dots can be, for example, a lattice of dots including hexagonal or square lattices as known in the art. The plurality of lines can form a grid, including perpendicular and parallel arrangements of the lines.

The dimensions of the individual patterns including dot diameters and the line widths can be, for example, about 1,000 nm or less, about 500 nm or less, about 300 nm or less, and more particularly about 100 nm or less. The range in dimension can be for example about 1 nm to about 750 nm, about 10 nm to about 500 nm, and more particularly about 100 nm to about 350 nm.

The number of patterns on the solid surface is not particularly limited. It can be, for example, at least 2, at least 3, at least 10, at least 100, at least 1,000, at least 10,000, even at least 100,000. Square arrangements are possible such as, for example, a 10×10 array.

The distance between the individual patterns on the solid surface, e.g., nanoarray, can vary. For example, the patterns can be separated by distances of less than one micron or more than one micron. The patterns can be parallel lines, perpendicular lines, arced curves, superimposed and overlapping shapes. The distances between the molecules can be from about 1 to about 20 nm for applications utilizing energy transfer (i.e., Forster radius). However, larger distances may be possible for applications utilizing enhancing polymers for chemiluminescence.

A variety of molecules, including, for example, proteins, polypeptides, oligopeptides, polymers, and fluorophores can be directly transferred and adsorbed to surfaces in a patterned fashion with use of nanolithographic printing, wherein the molecule is directly transferred from a tip such as, an atomic force microscope tip, to a substrate. Alternatively, however, in an indirect method, the nanolithographic printing can be used to deposit or deliver a compound in a pattern, and then the molecules can be assembled onto or adsorbed to the compound.

Solid surface of the present invention can be, for example, an insulator such as, for example, glass or a conductor such as, for example, metal, including gold. In addition, the solid surface can be a metal, a semiconductor, a magnetic material, a polymer material, a polymer-coated substrate, or a superconductor material. The solid surface can be previously treated with one or more adsorbates. Still further, examples of suitable solid surface include but are not limited to, metals, ceramics, metal oxides, semiconductor materials, magnetic materials, polymers or polymer coated substrates, superconductor materials, polystyrene, and glass. Metals include, but are not limited to gold, silver, aluminum, copper, platinum and palladium. Other solid surface onto which compounds may be patterned include, but are not limited to silica, silicon oxide, GaAs, and InP.

The solid surfaces can also include nanostructures, such as nanotubes, nanowires, and nanofibers. For example, carbon nanotubes or nanowires or nanofibers can be immobilized, synthesized or etched out of a surface. While these structures are attached to a surface, a patterning device such as, for example, DPN can be used to print molecules on to their surface. The assembly of patterns on these surfaces can occur by methods such as, for example, electrostatic layer-by-layer assembly.

The patterning compound can be, for example, chemisorbed or covalently bound to the substrate to anchor the patterning compound and improve stability. It can be, for example, a sulfur-containing compound such as, for example, a thiol, polythiol, sulfide, cyclic disulfide, and the like. It can be, for example, a sulfur-containing compound having a sulfur group at one end and a terminal reactive group at the other end, such as an alkane thiol with a carboxylic acid end group. The patterning compound can be a lower molecular weight compound of less than, for example, 100, or less than 500, or less than 1,000, or a higher molecular weight compound including oligomeric and polymeric compounds. Synthetic and natural patterning compounds can be used. Other examples include alkanethiols that have functional end-groups such as 16-mercaptohexadecanoic acid; hydrophobic thiols, such as 1-octadecanethiol; and organic coupling molecules, such as EDC and mannose-SH. Other examples of sulfur-containing compounds include, but are not limited to, hydrogen sulphide, mercaptans, thiols, sulphides, thioesters, polysulphides, cyclic sulphides, and thiophene derivatives. For instance, a sulfur-containing compound may comprise a thiol, phosphothiol, thiocyano, sulfonic acid, disulfide or isothiocyano group.

Other compounds include silicon-containing compounds that have a siloxy or silyl group that posseses a carboxylic acid group, aldehydes, alcohol, alkoxy or vinyl group. A compound can also possess an amine, nitrile, or isonitrile group. In one preferred embodiment, the solid surface will comprise sulfur adsorption on gold.

In general, the present invention uses nanolithographic methods, for example, dip pen nanolithographic printing, to assemble onto a surface at least two patterns of different molecules. The patterns can be provided, for example, in dots, or adjacent lines or stripes. The assembling process can be achieved by exposing the solid surface to a solution containing the desired molecules, e.g., immersing the solid surface into solution comprising the molecules; or spraying the solution onto the solid surface. Other methods of exposing the solid surface to a solution comprising the molecules including placing the solid surface in a chamber containing a solution vapor or mist, or pouring a solution comprising the molecules onto the solid surface. Alternatively, the assembling process can include depositing the molecules onto the solid surface using dip pen nanolithographic printing.

Non-specific binding of proteins to other, “non-compound” regions of a surface, can be prevented by covering, or “passivating,” those regions of the surface with another compound, or mixture of compounds, prior to exposure to the protein solution or sample (one or more passivating compounds). Known passivating compounds can be used and the invention is not particularly limited by this feature to the extent that non-specific adsorption does not occur. A variety of passivating compounds can be used including, for example, surfactants such as alkylene glycols which are functionalized to adsorb to the substrate. An example of a compound useful for passivating is 11-mercaptoundecyl-tri(ethylene glycol). After passivation, the resultant array can be called a passivated array.

A wide variety of assays exist which use light emission to determine the presence or concentration of a particular sample in a substance. The solid surface described herein can be used in any of these assays.

Assays employing the solid surfaces of the present invention can include conventional assays, such as protein and nucleic acid assays, DNA sequencing, ELISA, competitive assays, sandwich assays, agglutination types of assays, as well as other liquid phase and mixed phase assays.

In an exemplary embodiment, a solid surface of the present invention can be used in a energy transfer competitive assay format. In one example, a solid surface of the present invention specific for an analyte of interest and having a donor fluorophore and acceptor fluorophore separately patterned on it will be added to a sample. After incubation, the reaction mixture will be exposed to excitation light specific for the donor fluorophore. If a complex forms between the analyte and the binding agent, it will disrupt energy transfer from the donor to the acceptor molecule. Measurement of this light emission can provide an indication of the amount of analyte in the sample. Low analyte concentration will lead to a large signal whereas high analyte concentration will lead to a low signal.

In another example, a solid surface of the present invention specific for an analyte of interest will have a polymer patterned on it. The solid surface as well as a chemiluminescent enzyme conjugate that can bind to the analyte of interest will be mixed with a sample. The array will then be washed and exposed to the enzyme substrate. Measurement of the light can provide an indication of the amount of analyte in the sample.

These assay techniques provide the ability to detect both the presence and amount and concentration of small quantities of analytes and are useful in many application, for example medical diagnostics and forensic applications. Other applications include, for example, drug discovery, reporter-gene assays to monitor gene expression, second-messenger quantitation, protein kinase assays, antagonist/agonist screening, and protein-protein interaction analysis. As described, the present invention provides methods for detecting target nucleic acids in a sample. By “sample” is intended any sample obtained or derived from an individual, body fluid, cell line, tissue culture, or other source, including the environment, which contains or is suspected of containing the analyte of interest. As indicated, biological samples include body fluids (such as lymph, sera, plasma, urine, semen, expired air, synovial fluid and spinal fluid) and tissue sources. Methods for obtaining tissue biopsies and body fluids from mammals are well known in the art. Methods for obtaining environmental samples are also well known in the art.

Luminescence can be detected using conventional means, for example, a phomultiplier tube detector, camera luminometer, or even the naked eye. Luminescence intensity can be measured to determine the concentration of the substrate. The light emission of membrane-based assays can be imaged, for example, with standard x-ray film or Polaroid instant photographic film. Acquisition of digitized images can be accomplished, for example, with a phosphor storage screen and CCD camera instrumentation.

Luminescent signals generated in microplate-based assays can be quantified with a variety of commercially available luminometers. Such luminometers are normally instruments based on photomultiplier tube (PMT) technology, which move each well of the microplate directly below the PMT detector or the lens/fiber-optic light-collection interface. Sensitive detection of chemiluminescent signals in 96-well and higher-density microplates and microarrays is also possible with CCD camera instrumentation. Methods of detecting and quantitating luminescent labels are well known to those of skill in the art.

In an exemplary embodiment of the present invention, the analyte to be detected will be mRNA transcripts of a tumor antigen, such as prostate specific antigen (“PSA”). In one such embodiment, PSA mRNA can be detected by PSA-mRNA specific oligonucleotide probes linked to biotin. The biotin-linked probes will conjugate to a streptavidinylated linked solid surface of the present invention.

As also described, the present invention provides methods for detecting hormones in a biological sample. In an exemplary embodiment of the present invention, the analyte to be detected will be a hormone, such as thyrotropin (“TSH”). In one such embodiment, TSH can be probed by an anti-TSH antibody bound to biotin. The biotin-linked probes will conjugate to streptavidinylated linked solid surface of the present invention.

The present invention also provides kits for conducting an assay for the presence or concentration of an analyte. The kits of the present invention comprise a solid surface of the present invention.

In some embodiments, the kits will comprise a solid surface of the present invention separately patterned with a polymer for enhancing an alkaline phosphatase chemiluminescent reaction and a binding agent for an analyte of interest, alkaline phosphatase conjugate specific for the analyte of interest and a 1,2 dioxetane capable of interacting with the alkaline phosphatase conjugate. The kit can further comprise, for example, a hydrophobic fluorometric substrate for the enzyme, and optionally an enhancer molecule capable of improving fluorescence.

In some embodiments, the kits will comprise a solid surface of the present invention separately patterned with a polymer for enhancing a peroxidase chemiluminescent reaction and a binding agent for an analyte of interest, a peroxidase conjugate specific for the analyte of interest, a dihydropthalazinedione; and a peroxide compound in an amount which reacts with the dihydropthalazinedione in the presence of the peroxidase. The kit can optionally comprise any one or more of an enhancer compound in an amount which enhances light production from the dihydropthalazinedione in the presence of the peroxidase enzyme and/or decreases background chemiluminescence; a chelating agent in an amount which prevents the peroxide compound from activating the dihydropthalazinedione prior to reaction with the peroxidase; a chemiluminescence enhancing surfactant.

In some embodiments, the kits will comprise a solid surface of the present invention separately patterned with a polymer for enhancing a luciferase bioluminescent reaction and a binding agent for an analyte of interest, a luciferin conjugate specific for the analyte of interest, Mg; and ATP.

Although the foregoing invention has been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation. The disclosures of all publications, patents and patent applications cited herein are hereby incorporated by reference in their entirety and for all purposes.

### EXAMPLES

### Example 1

### Fabrication of an Antibody Nanoarray for Detecting the Prostate Tumor Marker, Prostate Specific Antigen (PSA) in Serum

Fabricate Arrays of Anti-PSA Antibody onto Gold Substrate

16-mercaptohexadecanoic acid (MHA) of the optimized feature sizes will be patterened onto a gold substrate (Piner et al., 1999) using DPN. Specifically, Si₃N₄ AFM tips (Nanolnk, Inc.; Chicago, Ill.) will create dot features, spaced 1 μm apart by contacting the gold substrate (Zhang et al., 2003). The diameter of the dot features will be determined by manipulating force and time of contact of the AFM tip, as previously described (Lee et al., 2004; Zhang et al., 2003). Any unprinted areas surrounding the features will be passivated by incubation with a 1 mM ethanolic solution of PEG-alkythiol (11-mercaptoundecyl-tri(ethylene glycol)) for 2 hours, followed by rinsing with ethanol and water (Lee et al., 2004).

Arrays will be designed to bind either total PSA or the separate fractions of complexed PSA-ACT or free PSA. Depending on the required specificity of the array, mouse monoclonal lgG antibodies for capturing either total or complexed PSA (Cat no. 10-P20; Fitzgerald Industries International; Concord, Mass.) or free PSA (Cat no. 10-P21) will be immobilized on the MIA dot features by incubation (1 hour) to produce an alternate pattern of anti-PSA. Unbound antibodies will be removed by rinsing with 10 mM PBS and Tween-20 solution (0.05%), and non-specific binding from plasma proteins prevented by passivation with BSA (10% solution in 10 mM PBS).

The topography of the absorbed manometer sized features of anti-PSA antibody on gold substrate will be determined by the NSCRIPTOR™ (Nanolnk, Inc.) in contact and lateral force modes. Typical measurements of lgG on the Y-aspect are approximately 6.5 nm in height (Lee et al., 2004; Zhang et al., 2003).

### Directly Detect Captured PSA Antigen

Samples containing the PSA antigen will be directly applied to the anti-PSA nanoarrays in an enclosed environment at room temperature for 1 hr. The samples will include purified free PSA (30-AP16; Fitzgerald International), complexed PSA (PSA-ACT, 30-AP13; Fitzgerald International), and human sera. After binding the antigen for 1 hr, the substrate will be rinsed with 10 mM PBS buffer with 0.05% Tween-20 solution. The binding of free PSA or complexed PSA will then be determined by AFM imaging (NSCRIPTORT™; Nanolnk, Inc.) in contact and lateral force modes. The base height of the capture antibody will be approximately 6.5 nm. The dimensions of PSA predicted by a homologous crystal structure are 7.2 nm×7.9 nm×4.5 nm (Carvalho 2002). Thus, the binding of free PSA will add approximately 4 nm to 8 nm in height, for a total height of between 10 to 15 nm. In contrast, the dimensions of alpha 1-antichymotrypsin (ACT) predicted by crystal structure are 7.5 nm×7.5 nm×20.8 nm (Chen et al., 2003). Thus, binding of complexed PSA with ACT would lead to a potential total topographic height of 17 nm to 35 nm (lgG+PSA+ACT).

### Assay PSA Antigen by Detection Antibody

To enhance the detection of PSA antigen, a sandwich assay will be performed with the binding of an additional PSA-specific detection antibody. The antibodies previously described for antigen capture have been paired by their manufacturer for use with specific detection antibodies in a sandwich format (total PSA: 10-P20; PSA-ACT: 10-P22; free PSA: 10-P20). After binding the antigen for 1 hr, the substrate will be rinsed with 10 mM PBS buffer with 0.05% Tween-20 solution. The washed substrate will then be incubated with detection antibody at 10 μg/ml in 10 mM PBS buffer solution for 45 minutes (Zhang et al., 2003). After binding the detection antibody for 1 hr, the substrate will be rinsed with 10 mM PBS buffer with 0.05% Tween-20 solution. The binding of the detection antibody will be determined by several methods including chemiluminescence based detection.

The binding of detection antibody will be characterized by imaging AFM as noted above. The additional topographic height of antigen antibody complex with detection antibody will be an approximate increase of 6.5 nm; this corresponds to the size of lgG antibody. For example, the height of a free PSA complex with capture and detection antibodies will be between 17 to 21 nm in height greater than substrate.

As an alternative, detection antibodies will be functionalized with biotin to facilitate subsequent luminescent-based detection. An lgG antibody biotinylation kit will be used to prepare biotinylated monoclonal antibody (Immunoprobe Biotinylation Kit, Cat No. BK-101; Sigma-Aldrich, St. Louis, Mo.). In brief, the detection antibody will be prepared at 1.0 mg/ml in a sodium phosphate buffer. This detection antibody solution will be added in a 1:10 dilution to BAC-SulfoNHS (biotinamidohexanoic acid 3-sulfo-N-hydroxysulfosuccinimide ester); BAC-SulfoNHS reacts with the free amino groups of the immunoglobulin forming stable amide bonds. This mixture is incubated at room temperature for 30 min and then purified over a Sephadex G-25 gel filtration column. The level of biotinylation of the eluted antibody will be determined by measuring the protein concentration (absorption at 290 nm) and the biotin content. Biotinylated detection antibodies can then be linked to a variety of labels; our primary label will be alkaline phosphatase (ALP). ALP can be readily linked to the biotinylated antibody by utilizing streptavidin conjugated ALP (Molecular Probes, Eugene, Oreg.).

### Example 2

### Fabrication of an Antibody Nanoarray for PSA Testing Comprising an Anti-PSA Antibody and a Chemiluminescent Signal Enhancement Grid for an Alkaline Phosphatase Label

### Fabricate Different Patterns of Anti-PSA Antibody and Enhancer Polymer

The DPN will be used to pattern 16-mercaptohexadecanoic acid (MHA) into the optimized feature sizes onto gold film to produce a nanoarray of negatively charged stripes. Any unprinted areas surrounding the features will be passivated by incubation with a 1 mM ethanolic solution of PEG-alkythiol (11-mercaptoundecyl-tri(ethylene gylcol)) for 2 hours followed by rinsing with ethanol and water. Mouse monoclonal lgG antibodies to PSA (anti-PSA) and poly[vinylbenzyl(benzyldimethyl ammonium)chloride] will be immobilized on the MHA dot features by incubation (1 hour) to produce an alternate pattern of anti-PSA and the polymer. Unbound antibodies will be removed by rinsing with 10 mM PBS and Tween-20 solution (0.05%), and non-specific binding from plasma proteins prevented by passivation with BSA (10% solution in 10 mM PBS).

### Standard Curve

A series of PSA standards (DPC Immulite) will be applied to separate antibody-polymer nanoarrays in an enclosed environment at room temperature. Binding of PSA to the nanoarray will be detected by AFM measurements using the DPN in imaging mode. This will confirm binding and give an AFM-generated standard curve for comparison purposes. The array will then be incubated with an optimized concentration of anti-PSA alkaline phosphatase conjugate (from DPC kit), washed and then exposed to the dioxetane substrate, AMPPD (Applied Biosystems). Light emission will be recorded using the NightOwl CCD camera, and the light emission related to PSA concentration and compared with the AFM results.

### Testing of Plasma Specimens

Standards (DPC Immulite) and plasma samples will be applied to the antibody nanoarrays (optimized spot size in previous experiment) in an enclosed environment at room temperature. All plasma samples and controls will incubated on the nanoarray for 1 hour at room temperature in an enclosed environment. Binding of PSA to the nanoarray will be detected by AFM measurements using the DPN in imaging mode. Results read off a calibration curve obtained with the PSA standards (DPC IMMULITE) will be compared with results obtained with our routine clinical PSA assay (DPC Immulite).

### Example 3

### Fabrication of Ordered Arrays of Energy Transfer Indicator Dyes and Binding Agents on a Solid Surface

Fabricate Arrays Printed with Donor and Acceptor Dye Inks

Alternate stripes of donor and acceptor dyes will be fabricated at a range of spacings between the stripes, starting with overlapping printing, then the smallest (currently 15 nm) spacing and increasing stepwise to 60 nm. This design will allow the degree of energy transfer with spacing to be determined so that the placement of the biotin can be optimized. The biotin must be placed between the donor and acceptor dye such that there is still an appreciable degree of energy transfer. However, when biotin binds to the streptavidin the energy transfer must be diminished.

### Donor or an Acceptor Dye Inks

The donor acceptor pair commonly used in energy transfer assays, fluorescein and tetramethylrhodamine, will be selected. Both are available with a long spacer arm (e.g., six carbon) terminating in a reactive functional group (e.g., Molecular Probes) and this provides the reactive moiety for attachment to the substrate surface when these molecules are used as the ink.

### Substrates

Gold is reactive towards thiol groups and an evaporated gold substrate (Nanolnk) will thus be used as the substrate. A silver island film will be prepared by reduction of silver ions in a thin film on a glass substrate surface using glucose (Lakowicz et al., 2001, 2002).

What is claimed:

1. An assay comprising:
a solid surface and, on the solid surface, at least two patterns of different molecules;
at least one of said patterns having a dimension between about 1 and about 1000 nanometers;
the geometry and identity of the molecules being such that interaction between them is modulated in the presence of an analyte.

2. The assay of claim 1 wherein at least one of said patterns has a dimension between 10 and 500 nanometers.

3. The assay of claim 1 wherein at least one of the molecules is a donor fluorophore and at least one of the molecules is an acceptor fluorophore, and the donor and acceptor fluorophores interact with each other to generate a detectable signal.

4. The assay of claim 3 further comprising a binding agent patterned on the solid surface.

5. The assay of claim 4 wherein energy transfer between the two fluorophores is disrupted when the analyte binds to the binding agent.

6. The assay of claim 1 wherein at least one of the molecules is an antibody and at least one of the molecules is a drug enzyme conjugate.

7. The assay of claim 1 wherein the at least one of the molecules is an antibody and at least one of the molecules is an antigen linked to an enzyme donor.

8. The assay of claim 1 wherein the molecules are patterned in alternate stripes.

9. The assay of claim 1 wherein the molecules are printed on the solid surface using molecular printing techniques.

10. The assay of claim 9 wherein the molecular printing technique is dip pen nanolithography.

11. The assay of claim 1 wherein the solid surface comprises two or more nanofibers twisted together.

12. The assay of claim 1 wherein the solid surface comprises a carbon nanotube.

13. An assay comprising:
a solid surface and, on the solid surface, at least two patterns of different molecules;
at least one of said patterns having a dimension between about 1 and about 1000 nanometers;
the geometry and identity of the molecules being such that one of said molecules modulates a signal generated by binding an analyte to the other of said molecules.

14. The assay of claim 13 wherein at least one of said patterns has a dimension between 10 and 500 nanometers.

15. The assay of claim 13 wherein at least one of the molecules is a polymer and at least one of the molecules is a binding agent adjacent to the polymer, and the polymer enhances a detectable signal generated in a bioluminescent or chemiluminescent assay for detecting binding of an analyte to the binding agent.

16. The assay of claim 13 wherein the molecules are printed on the solid surface using molecular printing techniques.

17. The assay of claim 16 wherein the molecular printing technique is dip pen nanolithography.

18. The assay of claim 13 wherein the solid surface comprises two or more nanofibers twisted together.

19. The assay of claim 13 wherein the solid surface comprises a carbon nanotube.

20. A method of constructing an assay comprising:
patterning upon a solid surface at least two patterns of different molecules;
at least one of said patterns having a dimension between about 1 and about 1000 nanometers;
wherein the different molecules interact with each other in the presence of the analyte to generate or modulate a detectable signal.

21. A method of constructing an assay comprising:
patterning upon a solid surface at least two patterns of different molecules;
at least one of said patterns having a dimension between about 1 and about 1000 nanometers;
wherein one of said molecules modulates a signal generated by binding of the other of said molecules to a target analyte.

22. A method for detecting the presence or absence of a target in a sample comprising exposing the assay of claim 1 to a sample.

23. A method for detecting the presence or absence of a target in a sample comprising exposing the assay of claim 13 to a sample.
